(19) United States
(12) Patent Application Publication
(10) Pub. No.: US 2006/0166347 A1
(43) Pub. Date: Jul. 27, 2006

(54) Sample preparation devices and methods

(75) Inventors: Konrad Faulstich, Fremont, CA (US); John R. Van Camp, San Ramon, CA (US); Charles Vann, El Granada, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/342,012

(22) Filed: Jan. 27, 2006

Related U.S. Application Data
(60) Provisional application No. 60/648,353, filed on Jan. 27, 2005.

(51) Int. Cl. C12N 1/06

(52) U.S. Cl. 435/259

(57) ABSTRACT

A method and device for preparing biological samples includes lysing the microorganisms, and collecting a desired biological material.

### RELATED APPLICATIONS

This application claims the benefits of priority to U.S. Provisional Application No. 60/648,353, filed Jan. 27, 2005, which is hereby incorporated by reference herein.

### FIELD

This disclosure is directed to sample preparation devices and methods and, more particularly, to apparatuses and methods for preparing samples for use in a microfluidic detection device, such as those used in the pharmaceutical and biotechnological fields.

### INTRODUCTION

Biological and chemical analysis techniques allow for precise measurements of minute quantities of sample materials. For liquid samples, sample preparation is a critical factor that determines the performance of analytical instrumentation. Some conventional sample preparation devices include filters that capture impurities and pass molecules of interest, while other devices include filters that retain molecules of interest and pass impurities. It can be desirable to combine various types of filters in a sample preparation device to separate impurities from the molecules of interest and prepare a biological sample for biological and/or chemical analysis. For example, it may be desirable to extract and purify nucleic acid (e.g., DNA or mRNA) from cells while also separating the nucleic acid from cell proteins that may inhibit follow-on chemistry and/or analysis such as PCR, for example.

Other conventional sample preparation techniques involve pelitizing nucleic acid using centrifugation of a lysed biological sample and washing away the supernant. Such techniques typically involve the use of relatively large-size equipment, including centrifuges that are relatively expensive. It may therefore be desirable to provide sample preparation devices and methods that are capable of separating nucleic acid or other biological material of interest from cells and/or proteins in a biological sample, wherein the devices for performing these functions are relatively small and/or relatively inexpensive. It may be further desirable to provide such sample preparation devices and methods that do not rely on centrifugation.

It also may be desirable to provide sample preparation and/or microfluidic detection devices that are configured for use in first responder settings, household environments, and/or physician offices, and/or are configured as consumable products.

### SUMMARY

Exemplary embodiments according to aspects of the present invention may satisfy one or more of the above-mentioned desirable features set forth above. Other features and advantages will become apparent from the detailed description which follows.

In various aspects, a method for preparing biological samples can include drawing a biological sample into a housing and flowing the sample through a first membrane configured to pass microorganisms not greater than a desired size and retain particles greater than the desired size. The method can further include retaining the passed microorganisms with a second membrane and drawing a lysis buffer to the second membrane. The method can also include drawing a lysate of biological material and an elution buffer to a third membrane and eluting the biological material from the third membrane.

In accordance with some aspects, a sample preparation device can comprise a housing and a member configured to selectively flow a biological sample in the housing in a first direction and a second direction, opposite to the first direction. The device can also comprise a first membrane in the housing in a path of the flow of biological material in the first direction, a second membrane in the housing in the path of the flow of biological material in the first direction, and a third membrane in the housing in the path of the flow of biological material in the first direction. The first membrane can be configured to pass microorganisms not greater than a desired size and retain particles greater than the desired size, the second membrane can be configured to retain microorganisms passed by the first membrane and a lysis buffer, and the third membrane can be configured to retain a lysate of biological material or fraction thereof and optionally an elution buffer.

In accordance with yet other exemplary aspects, a sample preparation device may include a housing configured to receive a biological sample. The housing may include a first chamber configured to mix a lysis buffer and the biological sample to form a lysate, a filtering mechanism configured to retain a biological material from the lysate, and a second chamber configured to contain an eluting buffer. The device also may include a flow member configured to provide force within the housing to cause liquid flow relative to the housing. The second chamber may be configured to be selectively placed in flow communication with the filtering mechanism to flow the eluting buffer to the filtering mechanism to elute the biological material from the filtering mechanism.

According to yet other exemplary aspects, a method for preparing a biological sample may include providing the biological sample and passing the biological sample through a first membrane adapted to select microorganisms according to size and retain particles greater than the desired size. The method may further include providing a lysis buffer to lyse the microorganisms and passing a biological material through a second membrane, wherein the second membrane retains the lysis buffer. The method also may include collecting the biological material with a third membrane and eluting the biological material from the third membrane. A housing may include the first membrane, the second membrane, and the third membrane

According to still further exemplary aspects, a method for preparing a biological sample may include mixing a biological sample with a lysing buffer to lyse microorganisms in the biological sample and create a lysate, flowing the lysate to a filtering mechanism configured to retain biological material in the lysate, and eluting the biological material from the filtering mechanism via an elution buffer. The mixing, flowing, and eluting may be performed within a multi-chambered syringe housing.

In yet various other exemplary embodiments, a sample preparation device may include means for holding a biological sample, means for lysing microorganisms in the biological sample to form a lysate, means for collecting a biological material from the lysate, means for eluting the biological material from the device, means for flowing liquid in the device, and means for isolating liquids in the device.

In some aspects, a microfluidic device comprises at least one capillary configured to receive a biological sample and direct the biological sample to at least one assay area, and a rheoline containing a desired amount of at least one liquid sample. The rheoline can be configured to direct the at least one liquid sample to the at least one capillary.

In the following description, certain aspects and embodiments will become evident. It should be understood that the invention, in its broadest sense, could be practiced without having one or more features of these aspects and embodiments. It should be understood that these aspects and embodiments are merely exemplary and explanatory and are not restrictive of the invention.

### BRIEF DESCRIPTION OF THE DRAWINGS

The drawings of this application illustrate exemplary embodiments of the invention and together with the description, serve to explain certain principles. In the drawings:

FIG. 1 is a cross-sectional side view of an exemplary sample preparation device in accordance with the present teachings;

FIG. 2 is a combination schematic and diagrammatic view of an exemplary biological detection system in accordance with the present teachings;

FIG. 3 is a combination schematic and diagrammatic view of an exemplary microfluidic device in accordance with the present teachings;

FIG. 4 is a combination schematic and diagrammatic view of an exemplary detection component of a microfluidic device in accordance with the present teachings;

FIG. 5 is a cross-sectional side view of an exemplary sample preparation device in accordance with the present teachings;,

FIG. 6 is a cross-sectional view of an exemplary sample preparation syringe in accordance with the present teachings;

FIG. 7 is a cross-sectional view of an exemplary embodiment of a sample preparation device in accordance with the present teachings; and

FIGS. 8A-8E schematically depict exemplary steps for using the sample preparation device of FIG. 7.

### DETAILED DESCRIPTION OF VARIOUS EMBODIMENTS

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Wherever possible, the same reference numbers will be used throughout the drawings to refer to the same or like parts.

The section headings used herein are for organizational purposes only, and are not to be construed as limiting the subject matter described. All documents cited in this application, including, but not limited to patents, patent applications, articles, books, and treatises, are expressly incorporated by reference in their entirety for any purpose. In the event that one or more of the incorporated literature and similar materials differs from or contradicts this application, including but not limited to defined terms, term usage, described techniques, or the like, this application controls.

Reference will now be made to various embodiments, examples of which are illustrated in the accompanying drawings. However, it will be understood that these various embodiments are not intended to limit the disclosure. On the contrary, the disclosure is intended to cover alternatives, modifications, and equivalents.

Aspects of the disclosure provide a sample preparation device configured to collect, concentrate, prepare, and load a biological sample for biological and/or chemical testing. Further aspects of the disclosure provide a biological detection device, for example, a handheld microfluidic device, configured to collect, concentrate, prepare, and load a biological sample for biological and/or chemical testing by the device.

A typical microdevice includes a substrate or body structure that has one or more microscale sample-support, manipulation, and/or analysis structures, such as a channel, well, chamber, reservoir, valve or the like disposed within it. As used herein, “microscale” refers to a fluid channel or conduit that has at least one cross-sectional dimension, e.g., width, depth or diameter, of less than about 1000 micrometers. In various embodiments, such channels have at least one cross-sectional dimension of no greater than 750 micrometers, and in some embodiments, from 1 to 500 micrometers (e.g., between 5 to 250, or between 5 to 100 micrometers). In one embodiment, a microscale channel has at least one cross-sectional dimension of between about 10-75 micrometers.

With respect to chambers or wells, “microscale,” as used herein, refers to structures configured-to hold a small (e.g., micro) volume of fluid; e.g., no greater than about 250-300 μl. In various embodiments, such chambers are configured to hold no more than 100 μl, no more than 75 μl, no more than 50 μl, no more than 25 μl, no more than 1 μl. In some embodiments, such chambers can be configured to hold, for example, about 30 μl.

A microdevice can be configured in any of a variety of shapes and sizes. In various embodiments, a microdevice can be generally rectangular, having a width dimension of no greater than about 15 cm (e.g., about 2, 6, 8 or 10 cm), and a length dimension of no greater than about 30 cm (e.g., about 3, 5, 10, 15 or 20 cm). In other embodiments, a microdevice can be generally square shaped. In still further embodiments, the substrate can be generally circular (i.e., disc-shaped), having a diameter of no greater than about 35 cm (e.g., about 7.5, 11.5, or 30.5 cm). The disc can have a central hole formed therein, e.g., to receive a spindle (having a diameter, e.g., of about 1.5 or 2.2 cm). Other shapes and dimensions are contemplated herein, as well.

The present teachings are well suited for microfluidic devices. The term “microfluidic” refers to a system or device having channels, chambers, wells, and/or reservoirs (e.g., a network of chambers and/or wells connected by channels) for supporting or accommodating very small (micro) volumes of fluids, and in which the channels, chambers, wells, and/or reservoirs have microscale dimensions.

The term “interior volume” as used herein refers to any structure, such as, for example, a sample region, channel, micro-fluidic channel, or chamber that provides containment for the a biological material either before, during, and/or after preparation. The interior volume can be bounded by a housing that can be opaque or transparent. Examples of housings can include cartridges which are complex and microfluidic or tubes that are simple and linear.

Further, the interior volume can take any shape including a well, a tube, a channel, a micro-fluidic channel, a vial, a cuvette, a capillary, a cube, an etched channel plate, a molded channel plate, an embossed channel plate, etc. The interior volume can be part of a combination of multiple interior volumes grouped into a row, an array, an assembly, etc. Multi-chamber arrays can include 12, 24, 36, 48, 96, 192, 384, or more, interior volume chambers.

The term “biological material” as used herein refers to any biological or chemical substance, alone or in solution, which is targeted for detection. The term “microorganisms” refers to cells that can be a component of an organism or an organism itself. The microorganisms can contain the biological material. The biological material can include one or more nucleic acid sequences to be monitored. The biological material can be monitored by polymerase chain reaction (PCR) and other reactions such as ligase chain reaction, antibody binding reaction, oligonucleotide ligations assay, and hybridization assay. The biological material can also be subjected to thermal cycling.

The term “filtering mechanism” as used herein may refer to a variety of structures used to filter, e.g., by size and/or type, one substance portion from another substance portion which passes through the filtering mechanism. Thus, various filtering mechanisms are described herein, including, for example, porous media, frits, beads, fibers, membranes, etc. Such filtering mechanisms also may include surface modified variants of these materials, as is described herein.

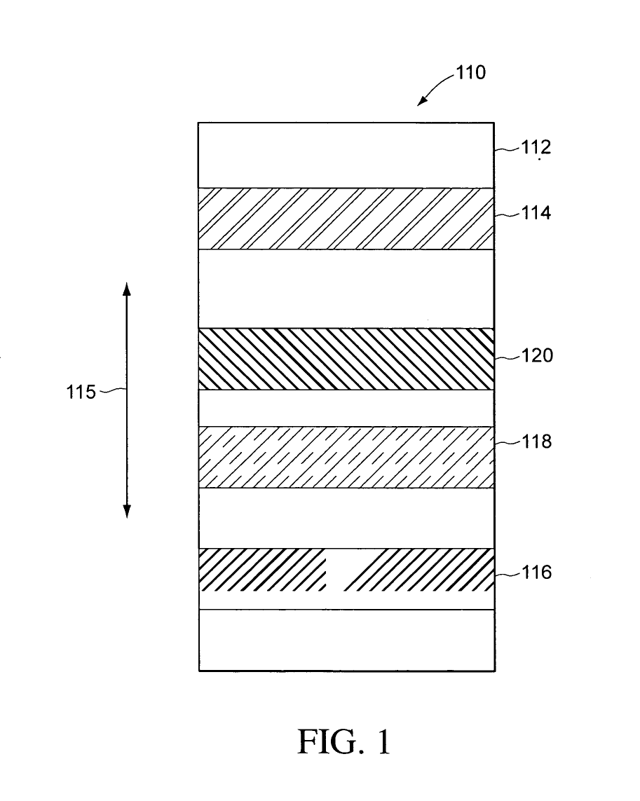

An exemplary embodiment of a sample preparation device 110 is shown in FIG. 1. The sample preparation device 110 can include a housing 112 forming an interior volume and a flow member 114 configured to generate a flow of a biological sample containing microorganisms in the housing 112 in first and second directions, the second direction being opposite to the first direction as illustrated by the double-headed arrow 115. The flow member 114 can be operationally controlled, for example, manually or automatically, to selectively flow the biological sample in either the first direction or the second direction. For example, the flow member 114 can be a piston such as, for example, a plunger of a syringe, as shown in FIG. 1. Alternatively, in some embodiments, the flow member 114 can be a reversible pump fluidly connected to the interior volume of the housing 112.

The device 110 can include a first membrane 116, a second membrane 118, and a third membrane 120 in the housing 112. The first, second, and third membranes 116, 118, 120, respectively, can be in a path of the flow of the biological sample. The first membrane 116 can be structured and arranged to pass microorganisms not greater than a desired size and retain particles greater than the desired size. The second membrane 118 can be structured and arranged to retain microorganisms passed by the first membrane 116 and a lysis buffer, and then to pass a lysate containing the biological material of interest that results from lysis of the microorganisms. The third membrane 120 can be structured and arranged to retain the lysate of biological material and optionally an elution buffer.

For example, a biological material can be prepared for testing by drawing the biological sample into the housing 112, for example, by manually or automatically operating the flow member 114 to flow the biological sample in the first direction (e.g., in the upward direction indicated by arrow 115 in FIG. 1). The flow member 114 can be operationally controlled to flow the sample in the first direction through the first membrane 116, which is configured to pass microorganisms not greater than a desired size and retain particles greater than the desired size. The passed microorganisms can then be maintained with the second membrane 118, and a lysis buffer can be drawn to the second membrane 118. The lysis buffer can be permitted to react with the retained microorganisms in the second membrane 118.

A lysate of the passed through microorganisms can be provided by maintaining the microorganisms in the lysis buffer. The lysate can be drawn in the first direction to the third membrane 120, along with an elution buffer. The third membrane 120 can be configured to retain the lysate of microorganisms or one or more components thereof and optionally a reagent or buffer. The biological material can then be eluted from the third membrane 120, for example, by controllably operating the flow member 114 to flow the lysate and elution buffer in the second direction (e.g., the downward direction of the arrow 115 in FIG. 1), for example, to a detection device. In an alternative aspect, the third membrane 120 may be configured to capture undesirable material from the lysate, such as, for example, a PCR inhibitor, and allow all other biological material to pass therethrough and be routed to a detection cell or the like for further processing.

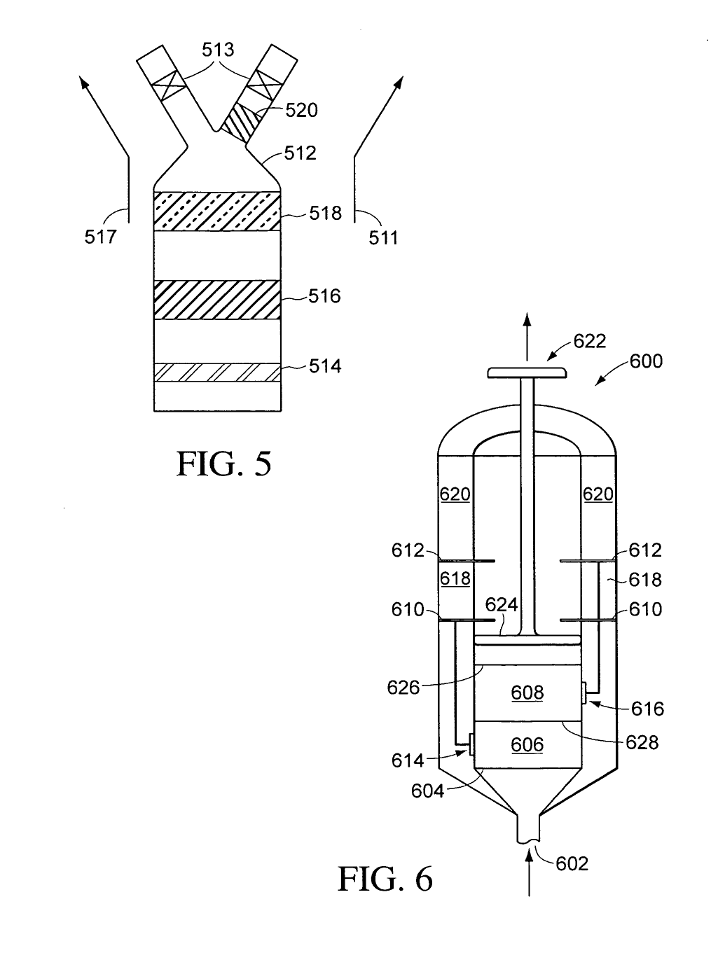

In various embodiments, as illustrated in FIG. 5, the housing 512 can include valving mechanisms 513 that prevent flow in the first direction 511 and flow in the second direction 517 to overlap. For example, the housing 512 downstream from the flow member 514 can be branched in a “Y” configuration with valves on each branch. The valves can be controllably operated in opposite states of one another such that when one valve is open and the other closed, flow down the elution path 517 is prevented during biological sample preparation down the membrane path 511. The flow member 514 can flow the microorganisms selected by size past the first membrane 516, flow the lysate past the second membrane, and capture the biological material with the third membrane 520 while flowing in the first direction 511. The flow member 514 can flow the biological material and elution buffer in the second direction 517 toward a detection device. Again, in an alternative aspect, the third membrane 520 may be configured to capture undesirable material from the sample while allowing other material (e.g., including the biological material of interest) to pass therethrough, for example, in the direction 511 to a detection cell or for further processing. Flow in the second direction 517 can then be used to remove the captured undesirable material from the third membrane 520 and route it to a waste collection area or the like.

In various embodiments, the first membrane 116 can be removed after selection of microorganisms by size. For example, particles greater than a desired size are retained and microorganisms not greater than the desired size are passed, then the retained particles and first membrane are removed. In various embodiments, the second membrane 118 can be removed after the lysate and elution buffer are drawn to the third membrane 120. Removal of the second membrane 118 can include removal of the lysis buffer. In some embodiments, the third membrane 120 can be removed after eluting the biological material from the third membrane 120. In various embodiments, the membranes can be isolated to prevent interference with flow in the first direction and/or second direction.

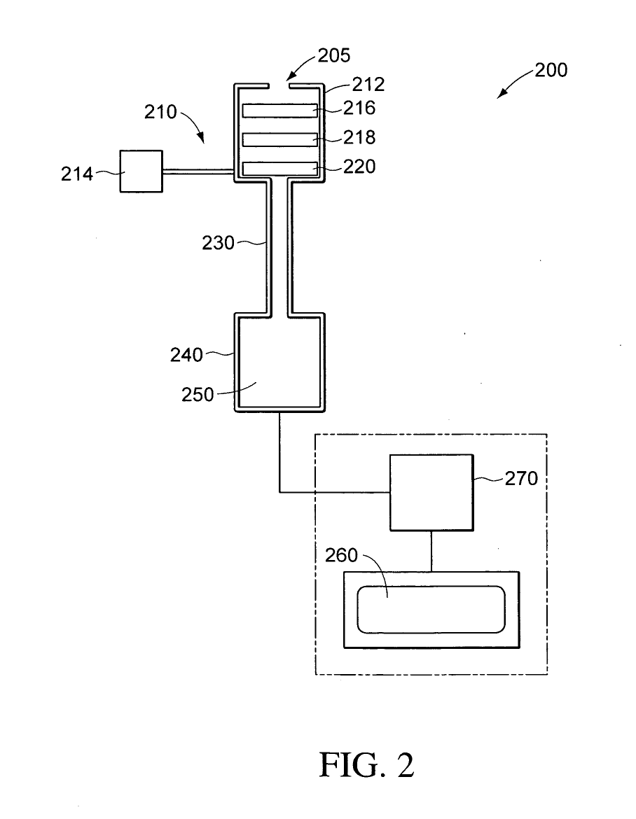

In various embodiments, a biological detection system 200, as shown for example in FIG. 2, can comprise an input port 205 fluidly connected to a sample preparation device 210, a channel 230 fluidly connected to the sample preparation device 210, and a detection cell 240 fluidly connected to channel 230.

The sample preparation device 210 can include a housing 212 and a flow member 214 configured to generate a flow of a biological sample in the housing 212 in first and second directions relative to the flow member 214, the second direction being opposite to the first direction. The flow member 214 can be operationally controlled, for example, manually or automatically, to selectively flow the biological sample in either the first direction or the second direction. For example, the flow member 214 can be a reversible pump fluidly connected with an interior of the housing 212, as shown in FIG. 2. Alternatively, in some embodiments, the flow member 214 can be a piston such as, for example, a plunger of a syringe, and located within the housing 212.

The sample preparation device 210 can include a first membrane 216, a second membrane 218, and a third membrane 220 in the housing 212. The first, second, and third membranes 216, 218, 220, respectively, can be in a path of the flow of the biological sample. The first membrane 216 can be structured and arranged to pass microorganisms not greater than a desired size and retain particles greater than the desired size. The second membrane 218 can be structured and arranged to retain microorganisms passed by the first membrane 216 and a lysis buffer, and to pass a lysate of biological material resulting the lysed microorganisms. The third membrane 220 can be structured and arranged to retain the lysate of biological material and an elution buffer.

In various embodiments, the first membrane 216 can be removed after particles greater than the desired size are retained and microorganisms not greater than a desired size are passed. In various embodiments, the second membrane 218 can be removed after the lysate and elution buffer are drawn to the third membrane 220. Removal of the second membrane 218 can include removal of the lysis buffer. In some embodiments, the third membrane 220 can be removed after eluting the biological material from the third membrane 220, for example, through channel 230 and to the detection cell 240.

As described above, the third membrane 220, in an alternative embodiment, may be configured to capture undesirable material and allow the passage of the remaining sample, including, for example, biological material of interest, through the channel 230 to the detection cell 240. The third membrane 220 may also be removed after passing the desired material to channel 230 to remove the captured undesirable materials.

The detection cell 240 can include various components configured to perform a detection technique based on the biological material of interest that is in interior volume 250. By way of example, the detection cell 240 may utilize detection techniques that rely on, such as, for example, chemiluninescence, bioluminescence, fluorescence, phosphorescence, colorimetry, electrochemical, and/or other suitable detection techniques, and may thus include components configured to perform those techniques. The biological detection system 200 can further include a display 260 that displays, for example, a data signal representative of light emitted in the interior volume 250. In various embodiments, the biological detection system 200 can further include a detector 270 optically connected or electrically connected to the detection cell 240 and the display 260. The detector 270 can be operative to process and convert, for example, the signal representative of the emitted light into the data signal that can be displayed on the display 260.

In various embodiments, a biological sample can be deposited into the input port 205 and directed to the sample preparation device 210. The biological material sample can be drawn into the housing 212 in a first direction, for example, by manually or automatically operating the flow member 214. The flow member 214 can be operationally controlled to flow the biological sample in the first direction through the first membrane 216, which is configured to pass microorganisms not greater than a desired size and retain particles greater than the desired size. The passed microorganisms can then be maintained with the second membrane 218, and a lysis buffer can be drawn to the second membrane 218. The lysis buffer can be permitted to react with the retained microorganisms in the second membrane 218.

A lysate of biological material resulting from the maintained microorganisms and the lysis buffer can be drawn in the first direction to the third membrane 220, along with an elution buffer. The third membrane 220 can be configured to retain the lysate of biological material or one or more components thereof and optionally a reagent or buffer. A prepared biological material can then be eluted from the third membrane 220, for example, by controllably operating the flow member 214 to flow the lysed biological material and elution buffer in the second direction relative to the flow member 214.

The prepared biological material can be directed to the detection cell 240 via the channel 230 and into the interior volume 250. One or more liquid samples can be combined with the prepared biological sample before, while, and/or after the prepared biological material is in the interior volume 250. As described above, the biological material can be detected by a variety of detection techniques, such as, for example, chemiluminescence, bioluminescence, fluorescence, phosphorescence, electrochemical, and/or other suitable detection techniques. In various exemplary embodiments, a reaction with the biological material can emit either a single or a narrow band of light, or the reaction can emit multiple wavelengths or multiple narrow bands of light. Moreover, in various embodiments, multiple biological materials can be received by the interior volume 250 producing at least a first wavelength and a second wavelength of light. In either case, when multiple wavelengths or multiple narrow bands are emitted, the detector 270 can collect the light by components such as, for example, a CCD, a photodiode, or a photomultiplier tube. The first wavelength and second wavelength can be resolved by filtering or a multi-wavelength detector, such as multi-layer CCD for multi-color detection.

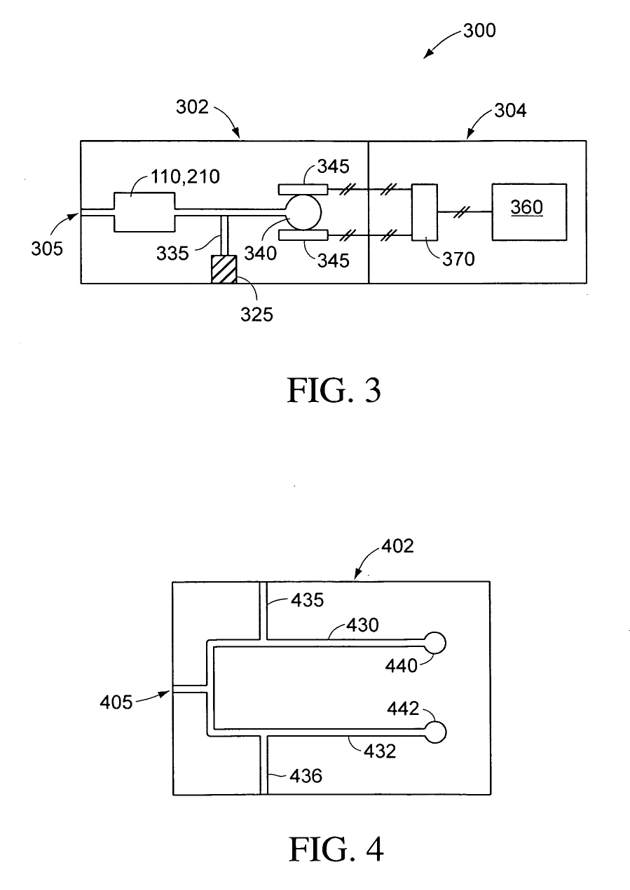

The biological detection system 200 described above with respect to FIG. 2 can be configured as a microfluidic device 300, for example, a handheld microfluidic device, as shown in FIG. 3. Referring to FIG. 3, the microfluidic device illustrated can include a detection component 302 electrically connected with a processing component 304. The detection and processing components 302, 304 can comprise a single integrated device of unitary structure, or they can be detachably connected and therefore separable from one another. For example, the detection and processing components 302, 304 can be manufactured separately and subsequently assembled together in a modular fashion. Thus, after a first biological sample is tested, the microfluidic device can be disassembled by separating the detection component 302 from the processing component 304 and a new detection component (not shown) can be coupled with the processing component 304 in order to test a second biological sample.

The detection component 302 can include one of the sample preparation devices 110, 210 described in detail above, or another sample preparation device known to those skilled in the art. In such embodiments, a biological material sample can be introduced to the detection component 302 via an inlet port 305 and prepared for testing by the sample preparation device 110, 210. The prepared biological material can then be eluted from the sample preparation device 110, 210 and directed to a detection cell 340 via a channel 330.

It should be appreciated that in various embodiments, the sample preparation device 110, 210 can be separate from the detection component 304, in which case the biological material sample can be prepared before being introduced to the detection component 302.

The detection component 302 can also include a liquid sample source 325 structured and arranged to supply a liquid sample to the detection cell 340 via channels 330, 335. Although FIG. 3 illustrates only one liquid sample source 325, it should be appreciated that the detection component 302 can include more than one liquid sample source and each source can provide the same or different liquid samples. It should be appreciated that one or more liquid samples can be supplied to the detection cell 340 before, while, and/or after the prepared biological sample is supplied to the detection cell 340.

In various embodiments, the liquid sample source 325 can be, for example, a rheological valve. Such valves permit a metered amount of liquid to pass when the proper shear force is applied to the valve. Shear force provides a turning or rotating motion to the valve. The line regulated by a rheological valve can be structured to contain a desired amount of a liquid sample, for example, in the picoliter to microliter range. The detection component 302 may be delivered to an end user with the rheologically regulated line pre-filled with the desired amount of liquid sample. The rheologically regulated line can include a valve-like structure arranged such that when the proper amount of shear force is provided, for example, by a rotational or translational force, the desired amount of liquid sample is introduced to the channels 335, 330 and supplied to the detection cell 340. Examples of rheologically regulated valves include those with spring loaded throttles or those with deformable walls or membranes to throttle flow based on the rheological properties of non-Newtonian fluids in the valve walls or changes in the rheological properties of Newtonian fluid as described, for example, in U.S. Pat. No. 6,158,270.

The detection component 302 can also include one or more detectors 345 proximal the detection cell 340. The detectors 345 can comprise a photosensitive material (not shown) such as, for example, a CCD structure, a photodiode, or a portion of a photomultiplier tube. In various exemplary embodiments, instead of or in addition to photosensitive materials, the detection component 302 may include excitation equipment configured to support fluorescence and/or phosphorescence detection techniques, as would be understood by those skilled in the art. Alternatively, the detection component 302 may include, in various embodiments, suitable equipment configured for use with electrochemical detection techniques, as would be understood by those skilled in the art.

The processing component 304 of the microfluidic device 300 can comprise a display 360 that displays, for example, a data signal representative of light emitted in the detection cell 340 and detected by the detectors 345. In various embodiments, the processing component 304 can further comprise a processor 370 electrically connected to the detectors 345 and the display 360. The processor 370 can be operative to process and convert, for example, the signal representative of the emitted light into the data signal that can be displayed on the display 360.

In various embodiments, a biological material sample can be deposited into the input port 305 and directed to the sample preparation device 110, 210. The sample preparation device 110, 210 can prepare the biological sample for testing, for example, by filtering and/or by chemical and/or biological reactions, as described above or as is well-known in the art. The prepared sample can then be eluted from the sample preparation device 110, 210 to the channel 330.

The biological sample can be directed to the detection cell 340 and combined with a liquid sample. As described above, when the biological material is in contact with the liquid sample, the combination may emit light. In various embodiments the combination can emit either a single or a narrow band of light, or the combination can emit multiple wavelengths or multiple narrow bands of light. Moreover, in various embodiments multiple biological materials and/or multiple liquid samples can be received by the detection cell 340. In either case, when multiple wavelengths or multiple narrow bands are emitted, they can be detected by the detectors 345.

For example, a first biological material in contact with a first liquid sample can produce a first wavelength. Similarly, a second biological material in contact with a second liquid sample can produce a second wavelength. Each of the first and second wavelengths can be optically coupled to the detectors 345, e.g., a photosensitive material, and they can be detected and resolved by the detection system. For example, the photosensitive material can generate a signal that is representative of the emitted light. Alternatively or additionally, corresponding signals may be generated via electrochemical, fluorescence, and/or phosphorenscence based detection techniques. In any case, the signal or signals generated can then be processed by the processor 370. The processor 370 then generates a data signal that can be displayed on display 360 in a visual format readable by a user.

Alternatively or additionally, the detection component 302 may be configured to be compatible with a USB port on a computer, PDA, or with a cell phone, which may serve as the processing component 304. In this way, data can be processed via these processing devices and may permit users to transmit results to a physician, pharmacy, medical lab, or the like. Further, it is envisioned that the processing component 304 may include software that provides suggested prescription drugs and/or other treatment options based on the analysis of organisms, nucleic acid, etc. in the detection component and other criteria, such as, for example, antibiotic resistance.

Referring now to FIG. 4, an exemplary detection component 402 can include one input port 405, two detection cells 440, 442, and two channels 430, 432 corresponding to the two detection cells 440, 442, respectively. Although no sample preparation device is shown in FIG. 4, it should be appreciated that the detection component 402 can include a sample preparation device, such as one of those shown in FIGS. 1 and 2.

The detection component 402 can include two liquid sample input ports 435, 436 associated with the two channels 430, 432, respectively. Accordingly, the same or different liquid samples can be added to each channel 430, 432 to facilitate multiple tests on the same or different biological samples. For example, the same biological sample can be supplied to both detection cells 440, 442, and each detection cell 440, 442 can be supplied with a different liquid sample via respective input ports 435, 436 to perform different assays based on nucleic acid and protein/antibodies or other biopolymers or compounds in parallel on the same detection component 402. By way of example only, one of the channels 430 or 432 may be provided with a reagent for performing an oligo-based test and the other channel 430 or 432 may be provided with a reagent for performing a protein-based test, thus providing a device capable of performing both a DNA and antibody test at the same time. Optionally, other cell-based test could also be performed. Alternatively, different biological samples can be supplied to the respective detection cells 440, 442 and the same or different liquid samples can be supplied to each cell 440, 442.

The detection component 402 can be connected to a processing component (not shown), similar to those described above in connection with FIG. 3. It should be appreciated that the processing component would need to be configured to be electrically connected to the detection component 402 and to provide the appropriate number of electrical connections corresponding to the number of detection cells 440, 442 and associated detectors (not shown).

It should be appreciated that that the channels 230, 330, 335, 430, 432, 435, 436 can be filled incrementally via multiple steps, thus removing the need to empty the channels after each step. It should also be appreciated that the detection cells can be filled via capillary forces in the channel. In these aforesaid systems, no pump, pressure, or vacuum would be necessary. Alternatively, the channels can be filled by providing a metered volume or providing a limited volume of liquid.

According to some embodiments, the processor 270, 370 and display 260, 360 can be about the size of analogous parts for conventional fever thermometers that include a processor and display. For example, the processor and display can be about 2 cm×2 cm.

In some embodiments, the detection device can be powered by a small battery or a pair of electrodes capable of generating a current. For example, a pair of electrodes can be configured to be placed into a moist soil to provide necessary power for field applications. In various embodiments, the electrodes can be intrinsic in the device and positioned to generate a current to power the device when the sample liquid bridges two electrodes made of different metals. Further, in exemplary embodiments, the current generated in these various embodiments can be used to heat the biological sample and/or liquid sample fluids to achieve desired temperatures for the enzymatic reactions. Further, ions contained in the sample liquid may initialize enzymatic reactions (e.g., may serve as cofactors).

In various embodiments, the sample preparation device can be a syringe, for example syringe 600 as illustrated in FIG. 6. Sample input 602 provides an entry for the biological sample. The inner volume of the syringe 600 can include a first membrane 604, a second membrane 628, and a third membrane 626. The first, second, and third membranes 604, 628, and 626, respectively, can be in the path of the flow of the biological sample. The first membrane 604 can be a size-exclusion membrane structured and arranged to pass microorganisms not greater than a desired size to first chamber 606 and retain particles greater than the desired size. The second membrane 628 can be a cell-capture membrane structured and arranged to retain microorganisms passed by the first membrane 604, and then to pass the lysate containing the biological material of interest that results from the lysis of the microorganisms to the second chamber 608 and retain the lysis buffer. The third membrane 626 can be a nucleic acid or protein binding membrane structured and arranged to retain the lysate of biological material and the elution buffer in second chamber 608. The movement of biological sample through the membranes in the first direction can be provided by moving plunger 622 in the direction of the arrow. Plunger 622 can include a shaft and plug 624. Plug 624 can provide the back pressure to draw the biological sample through the membranes and release the relevant buffers into first chamber 606 and second chamber 608. The syringe 600 can be constructed with an external volume in an outer cylinder relative to the inner volume that can include first reservoir 618 and second reservoir 620. First reservoir 618 can be fluidly coupled to first chamber 606 through first valve 614 and first seal 610. Second reservoir 620 can be fluidly coupled to second chamber 608 through second valve 616 and second seal 612. The movement of plug 624 past first seal 610 can break the seal and open first valve 614 to release the cell lysis buffer in first reservoir 618 into first chamber 606. The movement of plug 624 past second seal 612 can break the seal and open second valve 616 to release the nucleic acid or protein elution buffer in second reservoir 620 into second chamber 608.

In a manner similar to that described above With reference to FIGS. 1, 2 and 5, the third membrane 626, in an alternative embodiment, may be configured instead to capture undesirable material and allow the passage of the remaining sample, including, for example, biological material of interest and optionally an elution buffer. The material allowed to pass through the third membrane 626 may then be collected in the inner volume of the syringe 600 above the third membrane 626 and subsequently routed to a detection cell or the like for further processing. For example, once the desired material has passed through the third membrane 626, the contents of the chambers 606 and 608 and the membranes 604, 626, and 628 may be removed, for example, via a wash buffer or other mechanism. The desired material collected above the third membrane 626 may then be passed back through input 602 for further processing.

In various embodiments, the syringe can be replaced by a microfluidic channel with microfluidic seals and valves and pressure driven or capillary driven flow.

According to various exemplary embodiments, the membranes described herein can be separation membranes, size-exclusion membranes, and lysate membranes. Separation membranes can separate the biological material from the rest of the lysate by either capturing the biological material on the membrane or passing the biological material and retaining the substantial remainder of the lysate coming from the microorganisms. Lysate membrane can separate the lysate. Size-exclusion membranes can separate the microorganisms from particles of greater size in the sample.

According to various exemplary embodiments, the various membranes, and in particular the second and third membranes, described above in the exemplary embodiments of FIGS. 1, 2, 5, and 6 may be configured to be modifiable so as to further process and/or react with material (e.g., sample) passing therethrough). By way of example, at least a portion of the surface of one or more of the membranes may have one or more reagents, such as, for example, tethered lytic enzymes, affinity capture moieties, or other suitable reagents, bound (e.g., covalently or noncovalently) thereto.

Further, in lieu of the third membrane in the embodiments of FIGS. 1, 2, 5, and 6, it is envisioned that other filtering mechanisms configured to capture material (e.g., biological material of interest or undesirable material, such as, for example, PCR inhibitors) may be utilized, including, for example, a confined porous media bed, such as, for example, beads (e.g., silica, alumina, etc.), a frit, a bed of fibers (e.g., glass fibers), and or other suitable filtering mechanisms.

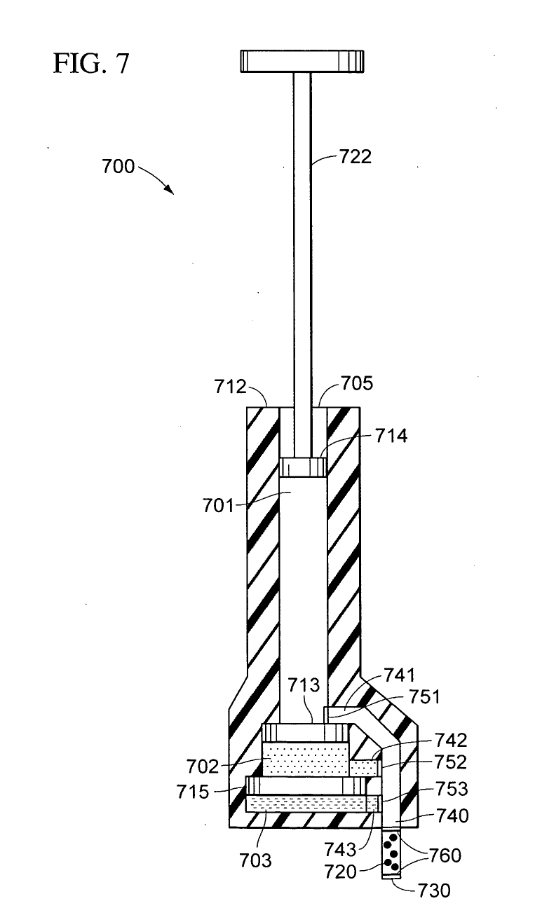

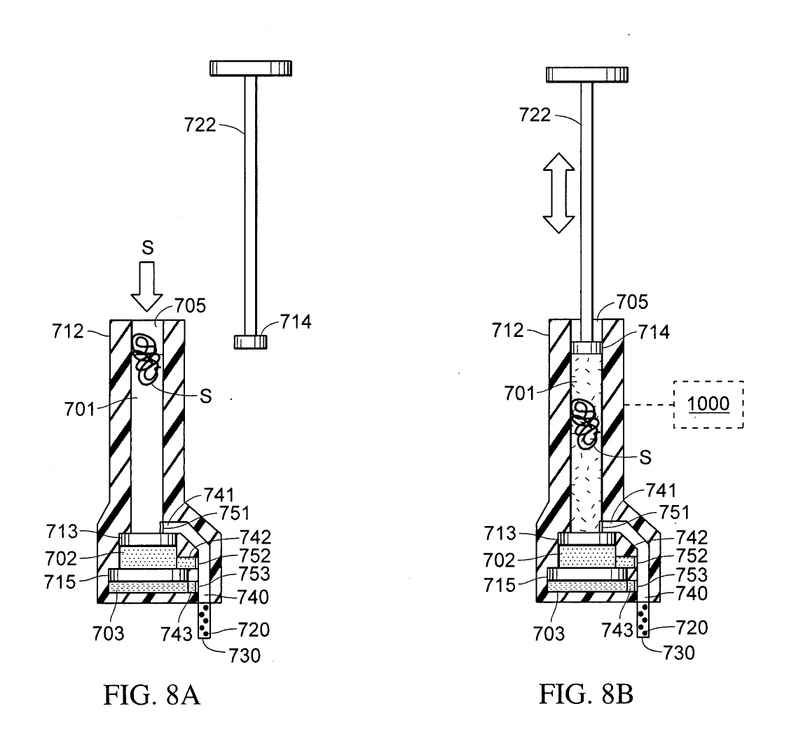

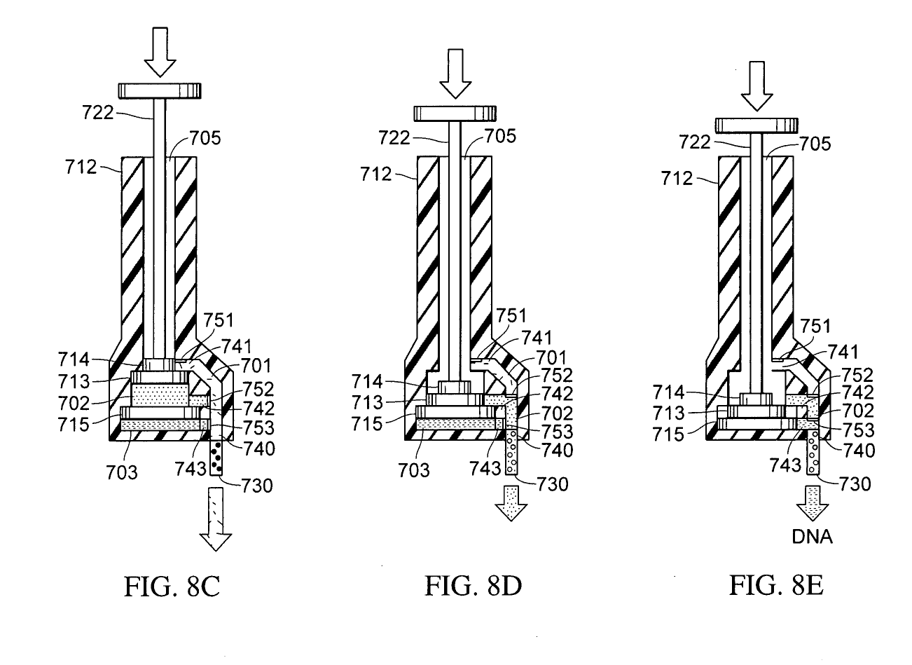

FIG. 7 depicts another exemplary embodiment of a sample preparation device in the form of a syringe-like structure. In the exemplary embodiment of FIG. 7, a sample preparation device 700 comprises a syringe body 712 comprising a housing defining three chambers 701, 702, 703. Chambers 701 and 702 are separated by a first movable isolation member 713 and chambers 702 and 703 are separated by a second movable isolation member 715. The movable isolation members 713 and 715 may be in the form of, for example, gaskets, plugs, or other similar members configured to separate the contents of the chambers. In the absence of a sufficient force acting on the isolation members 713 and 715, the isolation members 713 and 715 remain in place to separate (e.g., isolate the contents of) the chambers 701, 702, and 703 from each other. Upon applying a sufficient force, the isolation members 713 and 715 may be moved to cause the contents contained within a respective chamber 701, 702, and 703 to flow to different regions of the housing, as will explained in more detail below with reference to the description of FIGS. 8A-8E.

The syringe body 712 also defines an opening forming an input port 705 leading to the first chamber 701. The input port 705 is configured to receive a plunger mechanism 722 comprising a shaft and a piston 714 disposed at an end of the shaft that is inserted into the input port 705 and the first chamber 701. The plunger mechanism 722 is configured to be removable from the syringe body 712 so as to permit a biological sample to be loaded into the input port 705 and into the first chamber 701. As the plunger mechanism 722 is advanced within the chamber 701 (e.g., moved in a downward direction in FIG. 7), the piston 714 acts to increase pressure within the syringe body 712, thereby causing the contents (e.g., liquid) contained in the housing to flow in the housing, as will be described in more detail below.

The housing of the syringe body 712 further defines a channel 740 defining an opening forming an outlet 730. An end portion of the channel 740 proximate the outlet 730 may hold a filtering mechanism 720, such as, for example, a porous structure configured to capture (e.g., bind) biological material (e.g., nucleic acids, proteins, moieties, and/or other microorganisms) of interest flowing past the filtering mechanism 720, while allowing other material to pass therethrough. Suitable filtering mechanisms may include, for example, silica beads (as depicted in FIGS. 7 and 8), glass fibers, membranes, frits, and other similar structures made of various materials, including, for example, metals (e.g., alumina). Moreover, the filtering mechanisms may be surface modified variants of these materials, for example, by chemically binding (e.g., covalently or noncovalently) reagents to a surface thereof that may be used to process the sample. Those skilled in the art would understand how to select appropriate modifiers in order to achieve desired effects and/or filtering. In various exemplary embodiments, for example, when the filtering mechanism 720 is in the form of glass beads or similar porous media bed, a frit material 760, or other suitable porous structure, may be placed above and below the filtering mechanism 720 to hold the filtering mechanism 720 in position within the channel 740. In an alternative method to that described below with reference to FIGS. 8A-8E, after the appropriate material is captured by the filtering mechanism 720 and undesirable material has been washed via a washing buffer, the filtering mechanism 720 itself may be removed from the device 700 with the captured material thereon. The captured material may then be eluted from the filtering mechanism outside of the device 700. Alternatively the beads can be retained by coaxial capillaries of differing wall thickness to form a plug with a drain of sufficiently small size not to permit the beads to pass through.

Each of the chambers 701, 702, and 703 is configured to be selectively placed in flow communication with the channel 740 that leads to the output port 730. More specifically, each chamber 701, 702, and 703 is associated with a respective valve 751, 752, and 753 configured to selectively place the chambers 701, 702, and 703 in selective flow communication with the channel 740. Branch channels 741, 742, and 743 lead from each chamber 701, 702, and 703, respectively, to the channel 740.

According to various exemplary embodiments, each of the chambers 701, 702, and 703 is filled with various buffers prior to use of the device 700 for sample preparation. By way of example, the chamber 701 may contain a lysis buffer, the chamber 702 may contain a wash buffer, and the chamber 703 may contain an elution buffer. The isolation member 713 serves to separate the contents of the chamber 701 from the contents of the chamber 702 and the isolation member 715 serves to separate the contents of the chamber 702 from the contents of the chamber 703 prior to use of the device 700. Further, prior to use of the device, the valves 751, 752, and 753 serve to separate the contents of the chambers 701, 702, and 703, respectively, from the channel 740.

FIGS. 8A-8E schematically depict various exemplary steps to operate the sample preparation device 700, for example, to separate nucleic acid from cells in a biological sample. With reference to FIG. 8A, prior to use of the device 700, the various isolations members 713 and 715, and valves 751, 752, and 753, are in place within the syringe body 712 to separate the chambers 701, 702, and 703 from each other and from the channel 740, as described above. Further, chamber 701 contains a lysis buffer, chamber 702 contains a wash buffer, and chamber 703 contains an elution buffer. To begin sample preparation, the plunger mechanism 722 is removed from the syringe body 712 and a biological sample S is introduced into the chamber 701 via the input port 705.

After the sample S has been deposited into chamber 701, the plunger mechanism 722 is inserted into chamber 701, as illustrated in FIB. 8B, and the device 700 is manipulated to mix the sample S with the lysis buffer in chamber 701. By way of example, the syringe body 712 may be shaken and/or the plunger 722 may be advanced within the chamber 701 so as to cause the piston 714 to mix the sample S and the lysis buffer by moving the sample S in the chamber via pressure.

Alternatively or additionally, the sample and lysis buffer can be subjected to sonication by contacting chamber 701 with an ultrasonic transducer 1000 (shown in FIG. 8B in dotted line indicating that such a transducer is optional) to provide energy to penetrate the syringe body 712 and assist in the lysing of the microorganisms in the sample. According to various exemplary embodiments, the ultrasonic transducer may be in the form of a miniature ultrasonic horn coupled to the chamber 701. A solid phase (not shown) also may be provided in the chamber 701 to capture the components for lysing and the ultrasonic horn may focus the ultrasonic energy on the captured components on the solid phase. The solid phase may be a filter (e.g., a membrane or other filtering media) and may be configured to capture the sample components through size exclusion, affinity retention, or chemical selection, for example. Once the components are captured on the solid phase filtering mechanism, they may be lysed by transferring ultrasonic energy from the ultrasonic transducer to the captured components. Such sonication may be performed with or without the use of the lysis buffer in chamber 701.

Those having skill in the art would understand that the sonification techniques and devices described above could also be used in conjunction with the sample preparation device embodiments of FIGS. 1, 2, 3, 5, and 6 to perform the lysing functions in those embodiments, either alone or in combination with the lysing buffers discussed above with reference to those embodiments.

In various other embodiments, the sample and lysis buffer may be mixed by employing heat, which may be utilized alone or in addition to the other mixing techniques described above. In an exemplary aspect, a heating mechanism may be applied to the body 712, for example, to an external surface thereof.

After the biological sample and the lysis buffer have been sufficiently mixed so as to form a lysate, for example, the device has been manipulated for at least about 30 seconds according to various exemplary embodiments, the piston 714 may be further advanced (e.g., in a downward direction in FIG. 8C) in chamber 701 by pushing on the top portion of the plunger mechanism 722 that extends out of the syringe body 712. Eventually, as a result of increased pressure within the chamber 701 caused by advancing the piston 714 downward in the chamber 701, the valve 751 opens, as depicted in FIG. 8C. With the valve 751 open, the lysate formed from mixing the lysis buffer and the sample S in chamber 701 may flow into the channels 741 and 740, and through the filtering mechanism 720. The filtering mechanism 720 captures the nucleic acids (e.g., DNA, RNA, DNA+RNA), proteins, and/or other moieties or biological components (e.g., a PCR inhibitor) of interest contained in the lysate while permitting the rest of the mixture to exit from the channel 740 via the outlet 730. The entire contents of the chamber 701 may be emptied through the channel 740 by pushing on the plunger mechanism 722 until the piston 714 reaches the isolation member 713, as shown in FIG. 8C.

Once substantially all of the contents of chamber 701 have been emptied from the chamber 701 and the piston 714 impacts the isolation member 713, continued pushing on the plunger mechanism 722 causes the piston 714 to move the isolation member 713 in a downward direction and into the chamber 702, as shown in FIG. 8D. By advancing the isolation member 713 within the chamber 702, pressure in the chamber 702 increases due to the size and arrangement of the isolation member 713 and the chamber 702, thereby causing the valve 752 to open. The opening of the valve 752 places the branch channel 742 in flow communication with the channel 740, while simultaneously preventing flow communication between branch channel 741 and channel 740, as depicted by the positioning of the valve 752 in FIG. 8D. Thus, to the extent any of the lysate mixture remains in either branch channel 741 or chamber 701, it becomes isolated from the channel 740 due to the position of the valve 752 in FIG. 8D. As the plunger mechanism 722 continues advancement into the chamber 702, the contents of the chamber 702 (e.g., the wash buffer) may flow from the chamber 702, through the branch channel 742 and the channel 740, and out of the outlet port 730. As the wash buffer flows past the filtering mechanism (e.g., beads) 720, the wash buffer removes the lysis buffer but not the nucleic acid from the filtering mechanism 720. The plunger mechanism 722 may continue to be advanced until it contacts the isolation member 715 to flow substantially the entire contents of the chamber 702 into the channel 740.

By continuing to advance the plunger mechanism 722 in the downward direction, as depicted in FIG. 8E, the piston 714 also moves the isolation member 713 and the isolation member 715, which are in contact with each other, in a downward direction such that the isolation member 715 is advanced into the chamber 703. Due to the size and arrangement of the isolation member 715 and the chamber 703, movement of the isolation member 715 within the chamber 703 increases the pressure in the chamber 703, thereby opening valve 753 to place the branch channel 743 in flow communication with channel 740. Since the chamber 703 is in flow communication with the branch channel 743, the chamber 703 also is placed in flow communication with the channel 740 when the valve 753 is placed in the open position illustrated in FIG. 8E. At the same time, the position of valve 753 prevents flow communication between the branch channel 742 and the channel 740 below the position of the valve 753, to thereby isolate any contents remaining in branch channel 742 and chamber 702 from the filtering mechanism 720. Although FIG. 8E illustrates DNA being eluted from the syringe, alternatively, any nucleic acids or cellular (biological) components, e.g. RNA, cDNA, DNA, proteins, PCR inhibitors, etc, can be isolated according to the present teachings. Moreover, it is envisioned that the filtering mechanism 720 may be configured to capture material in the sample that is undesirable (e.g., impurities, PCR inhibitors, etc.) and permit the remainder of the sample containing biological material that may be of interest to further process to pass therethrough and be routed to an appropriate location for processing, such as, for example, a detection cell or the like. In this case, it may not be necessary to provide a chamber an isolation member containing an elution buffer, but rather only the washing buffer chamber and corresponding isolation member to permit washing of undesirable substances captured by the beads.

Advancement of the isolation member 715 within the chamber 703 causes the elution buffer contained in the chamber 703 to flow through the branch channel 743, the channel 740, past the filtering mechanism 720, and out of the outlet 730. The elution buffer removes the nucleic acid from the beads so that it flows with the elution buffer out of the output port 730 and may be directed to a detection component. By way of example only, the device 700 may replaced the sample preparation devices 110, 210 shown in FIG. 3 and the output port 730 may be in flow communication with a channel 330 or the like to flow the nucleic acid and/or other compounds of interest to the detection component 340.

The sample preparation device according to the exemplary embodiment of FIG. 7 may provide advantages over conventional sample preparation devices. For example, the device of FIG. 7 may be provided in the form of a consumable (e.g., disposable) device that is relatively inexpensive to manufacture and relatively easy to operate. Further, the sample preparation device of FIG. 7 is able to purify nucleic acid from cells while removing undesirable proteins, etc., and also provides for the concentration of nucleic acid or other captured biological material of interest into a relatively small volume, which may permit the volume of the elution buffer to be substantially smaller than the starting sample volume introduced to the sample preparation device. By way of example only, chamber 701 may have a volume ranging from about 1 microliter to about 10 microliters and the filtering mechanism 720 may result in a collection volume of about 10 microliters. The elution buffer may be selected to allow the desired biological material to be eluted from the beads in a relatively smaller volume, thereby yielding a sample with a relatively higher concentration of the purified target molecule(s).

In various exemplary embodiments, the sample preparation devices of FIGS. 5-7 also may be used in combination with the detection and processing components of FIGS. 2 and 3. For example, the sample preparation devices of FIGS. 5-7 may be used to supply prepared sample to the detection components 240 or 340 of FIGS. 2 and 3. Moreover, the devices of FIGS. 5-7 may be configured as microfluidic devices having mircrofluidic channels and the like, and incorporated as an integral part of a microfluidic biological detection system, such as, for example, biological detection system 200 or 300.

In various embodiments, the lysis buffer can be replaced with or used in conjunction with physical lysis methods to lyse the microorganisms. The physical lysis methods can include sonic, thermal, and ballistic lysis. The second membrane can then pass the lysate without having to retain any lysis buffer.

In various embodiments, the first membrane can include other selection criteria other than size-exclusion. Other selection criteria membranes can include affinity capture membranes, for example hapten mediated capture, and ion-exchange membranes, for example selective capture of moieties based upon isoelectric points.

In various embodiments, at least one of the membranes can be replaced by alternative purification methods such as a bed of porous media, for example silica, providing the same sample preparation as the membrane.

When referring to various directional relationships herein, such as, for example, downward, upward, etc., such relationships are referred to in the context of the orientation of the drawings. It should be understood, however, that the devices in actuality may be oriented in directions other than those illustrated in the drawings and directional relationships would by altered accordingly.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “less than 10” includes any and all subranges between (and including) the minimum value of zero and the maximum value of 10, that is, any and all subranges having a minimum value of equal to or greater than zero and a maximum value of equal to or less than 10, e.g., 1 to 5.

It is noted that, as used in this specification and the appended claims, the singular forms “a,” “an,” and “the,” include plural referents unless expressly and unequivocally limited to one referent. Thus, for example, reference to “a biological” includes two or more different biological samples. As used herein, the term “include” and its grammatical variants are intended to be non-limiting, such that recitation of items in a list is not to the exclusion of other like items that can be substituted or added to the listed items.

It will be apparent to those skilled in the art that various modifications and variations can be made to the sample preparation device and method of the present disclosure without departing from the scope its teachings. Other embodiments of the disclosure will be apparent to those skilled in the art from consideration of the specification and practice of the teachings disclosed herein. It is intended that the specification and examples be considered as exemplary only.

What is claimed is:

1. A sample preparation method, the method comprising:
providing a biological sample, wherein the biological sample comprises microorganisms;
providing a housing to lyse the microorganisms and separate biological material contained in the microorganisms, wherein the housing comprises a separation membrane to separate the biological material contained in the microorganisms;
providing a lysis buffer to lyse the microorganisms;
lysing the microorganisms to release the biological material and form a lysate;
collecting the biological material.

2. The method of claim 1, wherein the housing further comprises a lysate membrane and the method further comprising:
passing the lysate through the lysate membrane.

3. The method of claim 2, wherein the lysate membrane comprises one or more reagents configured for processing the sample, wherein the reagent is chemically bound to a surface portion of the membrane.

4. The method of claim 2, wherein the housing further comprises an exclusion membrane and the method further comprising:
passing the biological sample though the exclusion membrane to select the microorganisms and exclude other particles.

5. The method of claim 1, the method further comprising:
eluting the biological material from the separation membrane.

6. The method of claim 5, wherein the housing further comprises a flow member adapted for eluting the biological material from the separation membrane.

7. The method of claim 1, the method further comprising:
supplying ultrasonic energy to the housing to lyse the microorganisms.

8. The method of claim 1, wherein the housing has a tube-like configuration.

9. The method of claim 1, wherein the housing further comprises a rheologically regulated valve adapted to provide metered amounts of liquid to the housing.

10. The method of claim 7, wherein the Theologically regulated valve comprises a spring-loaded throttle, a non-Newtonian fluid, or a Newtonian fluid with an additive adapted to change the rheological properties of the Newtonian fluid.

11. The method of claim 1, the method further comprising:
supplying physically manipulation or heat to the housing to lyse the microorganisms

12. A sample preparation method, the method comprising:
providing a housing configured to receive a biological sample, the housing comprising:
a first chamber configured to mix a lysis buffer and the biological sample to form a lysate;
a filtering mechanism configured to retain a biological material from the lysate;
a second chamber configured to contain an eluting buffer, the second chamber being configured to be selectively placed in flow communication with the filtering mechanism to flow the eluting buffer to the filtering mechanism to elute the biological material from the filtering mechanism; and
providing a flow member configured to provide force within the housing to cause liquid flow relative to the housing
providing the biological sample to the first chamber, wherein the biological sample comprises microorganisms;
lysing the microorganisms to release the biological material and form a lysate; and
eluting the biological material from the filtering mechanism.

13. The method of claim 12, wherein the filtering mechanism comprises beads.

14. The method of claim 12, wherein the filtering mechanism comprises a membrane.

15. The method of claim 12, wherein the housing comprises a syringe housing.

16. The method of claim 12, the method further comprising:
providing an ultrasonic transducer configured to apply ultrasonic energy to at least a portion of the housing.

17. The method of claim 12, wherein the flow member is configured to provide a pressure force in the housing to selectively open at least one valve to cause liquid flow through the at least one valve.

18. The method of claim 12, wherein the housing further comprises a third chamber configured to contain a wash buffer, the third chamber being configured to be selectively placed in flow communication with the filtering mechanism to flow the wash buffer to the filtering mechanism.

19. The method of claim 12, wherein the first chamber is configured to be selectively placed in flow communication with the filtering mechanism to flow the lysate to the filtering mechanism.

20. A method for preparing a biological sample, the method comprising:
mixing a biological sample with a lysing buffer to lyse microorganisms in the biological sample and create a lysate; and
flowing the lysate to a filtering mechanism configured to pass biological material in the lysate and capture other material in the lysate,
wherein the mixing and flowing are performed within a multi-chambered syringe housing.
